(19) United States
(12) Patent Application Publication
(10) Pub. No.: US 2017/0119167 A1
(43) Pub. Date: May 4, 2017

(54) Spinal Support Pillow

(71) Applicant: George Madjitey, Sugar Land, TX (US)

(72) Inventor: George Madjitey, Sugar Land, TX (US)

(21) Appl. No.: 14/931,688

(22) Filed: Nov. 3, 2015

(51) Int. Cl. A47C 20/02; A61F 5/01; A47G 9/10

(52) U.S. Cl. CPC A47C 20/027; A47G 9/10; A61F 5/01; A47G 2009/1018

(57) ABSTRACT

The present invention discloses a stress relieving back supporting pillow. The present invention provides a method to relieve stress on the back. The present invention provides a device as an alternative method of preventing back pain. A therapeutic pillow with opposing attached retainers to secure the person in a flat position while sleeping. The pillow includes a cover with an inner core made of firm material to support the spine of the lower back. The inner core includes a top portion with an attached polyhedron shaped base with a flat bottom side to support the pillow on a flat surface of a mattress. The corners of the base can taper downwardly providing more support. The pillow support securely attaches to the bed.

### BACKGROUND

The complaint of back pain is among the most common medical conditions. Back pain can be among the most difficult and frustrating problems for patients and their doctors. Because back pain is sometimes difficult to treat, a better understanding of the cause of your condition will assist in your recovery. Muscle strains are the most common cause of low back pain. Arthritis most commonly affects joints such as knees and fingers. However, arthritis can affect any joint in the body, including the small joints of the spine. Arthritis of the spine can cause back pain with movement. Patients often find help from ice, heat, and medications. If the basic treatments for back pain do not relieve your symptoms, the next step is to seek medical evaluation. Rest, ice, heat, exercises, medications, and other treatments may all be useful in relieving your symptoms.

Back pain lower down in the spine can occur for a number of different reasons. The most common causes include:
Injury. Many people get a low back strain when they lift a heavy item incorrectly. They may also injure their backs while playing sports or during a motor vehicle.
Repetitive stress. When people have jobs that require repetitive lifting and twisting, they deliver a lot of wear and tear to their spinal columns.
Spinal disc problems. Many people have bulging or herniated spinal discs, but they experience no symptoms. However, if a bulging or herniated disc starts to press on a spinal nerve, they may notice significant low back pain or other related problems.
Arthritis. Arthritis causes the cartilage inside of the spinal joints to wear out. The cartilage loss can lead to pinched nerves or to other sources of low back pain.

Treatments range from conservative options, like medication and physical therapy for acute back pain and surgical procedures for more chronic low back pain that are caused by more serious conditions like disc related injuries. Physical therapy can range from exercise movements, heat therapy to massage therapy. There is evidence that practicing correct posture helps prevent and alleviate lower back pain. This evidence applies to sleeping postures as well. The use of devices such as pillows can help prevent and alleviate lower back pain by maintaining correct spinal alignment during sleep.

The prior art discloses various support pillows and cushions. U.S. Pat. No. 4,459,714 discloses a cushion filled with air or water. The plurality of cushions are hooked together. U.S. Pat. No. 4,777,678 discloses triangular shaped support pillows that are interconnected. U.S. Pat. No. 4,794,657 discloses a pillow assembly with head, trunk and leg pillow compartments. U.S. Pat. No. 5,479,667 discloses an ergonomic pillow assembly that has retainers that maintain a person on the pillow, but is not removable. U.S. Pat. No. 4,876,755 discloses a removable pillow assembly that supports the head and back. U.S. Pat. No. 5,987,678 discloses a full spinal support pillow. U.S. Pat. No. 6,170,103 discloses a support pillow. U.S. Pat. No. 7,634,829 discloses a head and chest support kit where a person sleeps head first. U.S. Pat. No. 8,671,480 provides abdominal and back support pillow for pregnant women.

### SUMMARY

In accordance with the present invention, there is provided an orthopedic pillow assembly which maintains a person's body in a flat position while sleeping. One object of this invention is to provide an ergonomic pillow assembly which provides support necessary to the user's spine namely in cervical, thoracic and lumbar sections to prevent the spine from flexing to the right or left while one is sleeping reclining on either the right or left side. The lumbar section of the spine consists of a group of live vertebrae extending down from the thoracic section of the spine assembly to the sacrum, commonly referred to as the lower back.

One object of this invention is to provide an ergonomic pillow assembly allowing for ample range of motion of the user's lower arm and shoulder while sleeping. An additional object of this invention is to provide an ergonomic pillow assembly which eliminates problems associated with conventional pillows which move out of position as a user changes positions while sleeping. The design of the ergonomic pillow assembly is simple with no moving parts or sections which must be reconfigured to obtain the complete utility thereof. Still, another object of this invention is to provide an ergonomic pillow assembly that is economical to manufacture; simple to use; achieving the ideal support to the user's torso; and substantially maintenance free.

### DESCRIPTION OF DRAWINGS

Various other objects, advantages, and features of this invention will become apparent to those skilled in the art from the following discussion, taken in conjunction with the accompanying drawings, in which:

FIG. 1 is a front perspective view of the present invention.

FIG. 2 is a perspective view of the present invention with a person using the pillow and retainers.

FIG. 2A is a side perspective view of present invention with a person lying on the pillow.

FIG. 3 is a front view of opposing retainer.

FIG. 4 is a back view of opposing retainer.

FIG. 5 is a side view of the retainer.

FIG. 6 is a side view of the retainer.

FIG. 7 is a side view of the center pillow with square retainers.

FIG. 8 is a side view of the center pillow with rounded retainers.

FIG. 9 is a side view of the center pillow with triangular shaped retainers.

FIG. 10 is a front perspective view of the adjusting Velcro to widen the retainers.

FIG. 11 is a back perspective view of the Velcro to widen the retainers.

### DETAILED DESCRIPTION

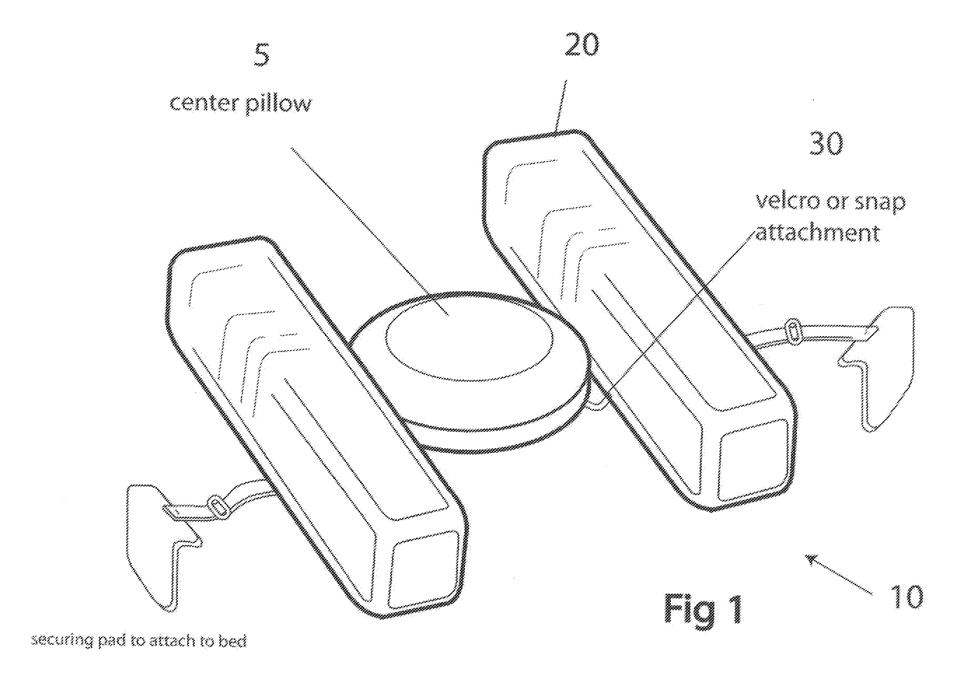

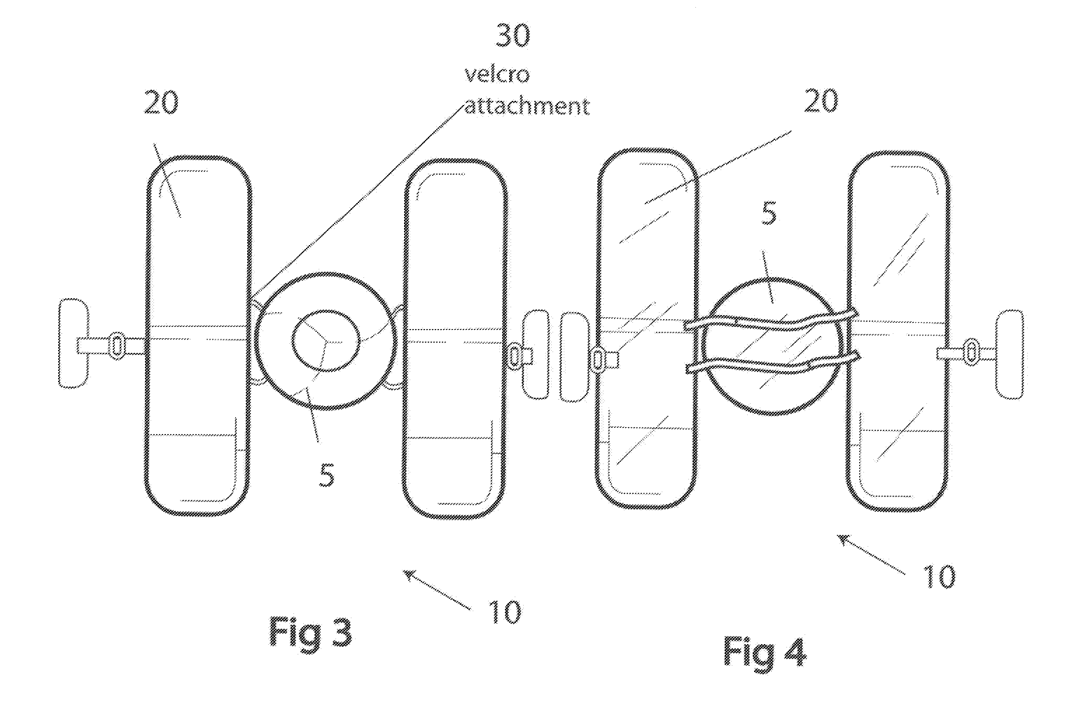

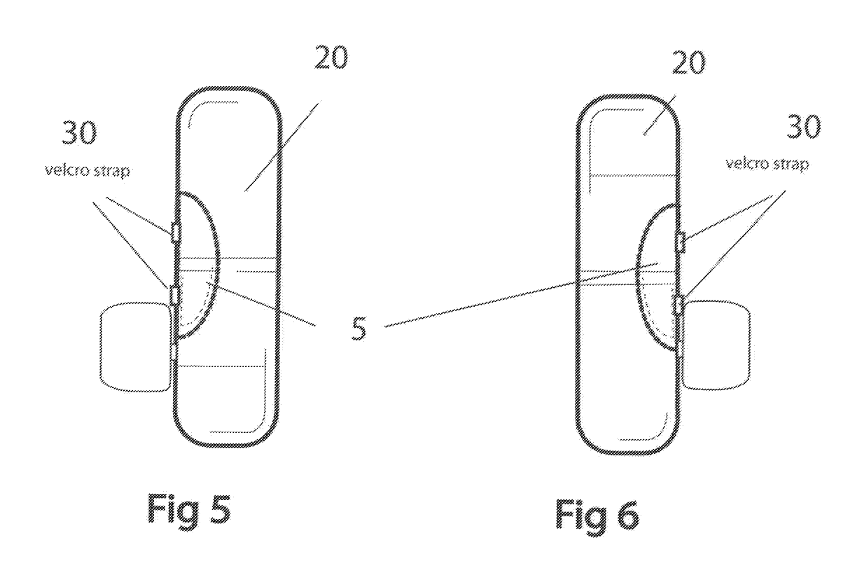

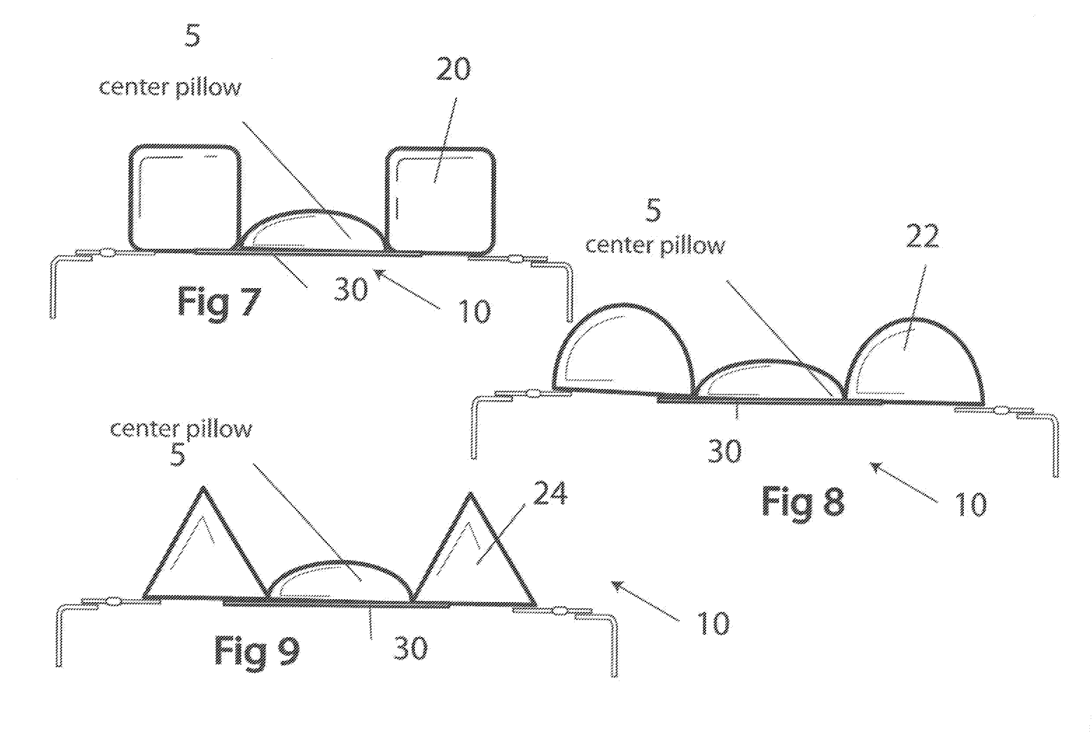

With reference now to the drawings, a new ergonomic back support assembly embodying the principles and concepts of the present invention is disclosed. Referring to FIG. 1 to 6, there is shown the preferred embodiment of the present invention which comprises a pillow (5) with opposing fasteners (30, 31) attached thereto. Ergonomic pillow (5) is disposed between opposing retainers (20, 21) and attached repeatedly to opposing retainers (20, 21). The entire back support assembly is secured to bed by fasteners (35, 36).

Low back pain causes 40% of missed days of work in the United States. Additionally, it is the single leading cause of disability worldwide. While standing upright in an orthostatic position, the human body is held in an upright position supported by the feet. In this upright position, the spinal column is also in a suspended straight state.

As shown, trunk or torso (33) is the central part of the human body which extends between the neck and limbs. The most critical organs are housed within the torso. The human back is the large posterior area of the human body, rising from the top of the buttocks to the back of the neck and the shoulders. As shown the torso (33) is disposed opposite to the chest with a height being defined by the vertebral column (commonly referred to as the spine or backbone). The central feature of the human back is the vertebral column (32) extending the length from the top of the thoracic vertebrae to the bottom of the lumbar vertebrae, housing the spinal cord in its spinal canal, and forming the curvature of the lower back.

The lower back portion also called the lumbar vertebrae has the five vertebrae between the rib cage and the pelvis that supports the are within the back. The pillow of the present invention is design to protect the are within the lower back. The lumbar vertebrae help supports the weight of the body, and permit movement when the human body is in the upright position. The purpose of the present invention is to support the arc in the lower back to the spine therein.

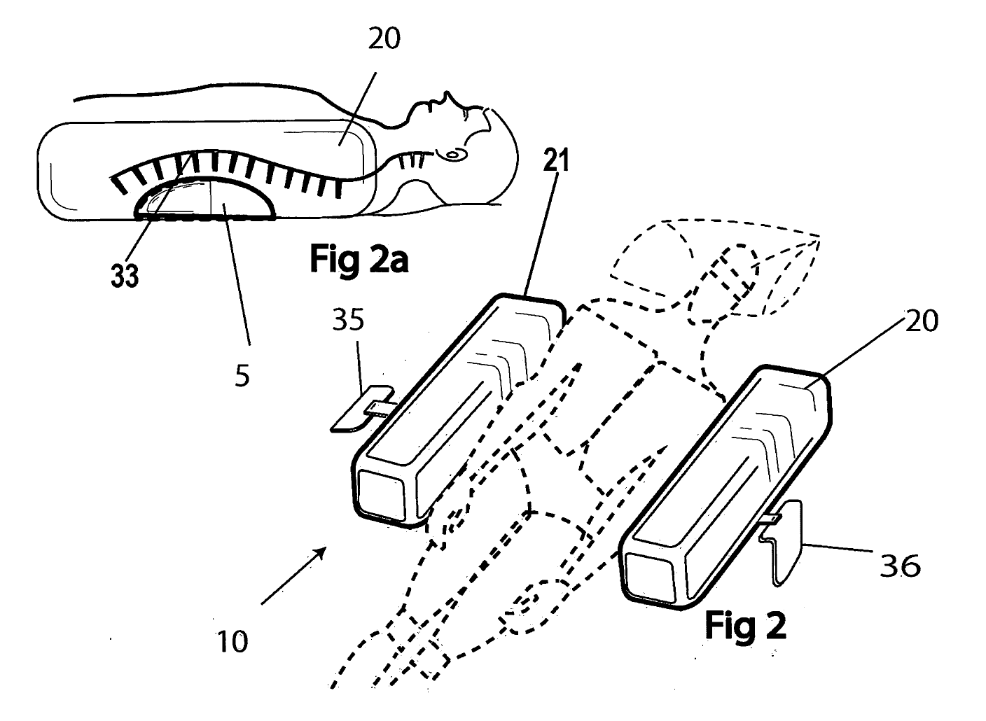

As shown in FIG. 2, the user can be appropriately positioned and supported by placing themselves between me opposing retainers (20, 21). The pillow (5) has a depth designed specifically to support the diameter of the torso back curve (33) of the user. While sleeping the pillow (5) pushes the spinal cord in a straight suspended state thereby reducing the strain on the spine.

The pillow (5) has an inner core encased by a covering having the same shape and configuration as the body of the pillow (5). Pillow (5) of the present invention is constructed of firm foam like material or another suitable material. When the opposing retainers (20, 21) are connected, a support area is formed wherein the user can comfortably lie without moving out of the optimal positioning as shown in (1). The retainers (20, 21) have an inner depth allowing the body to be retained within the support area while sleeping.

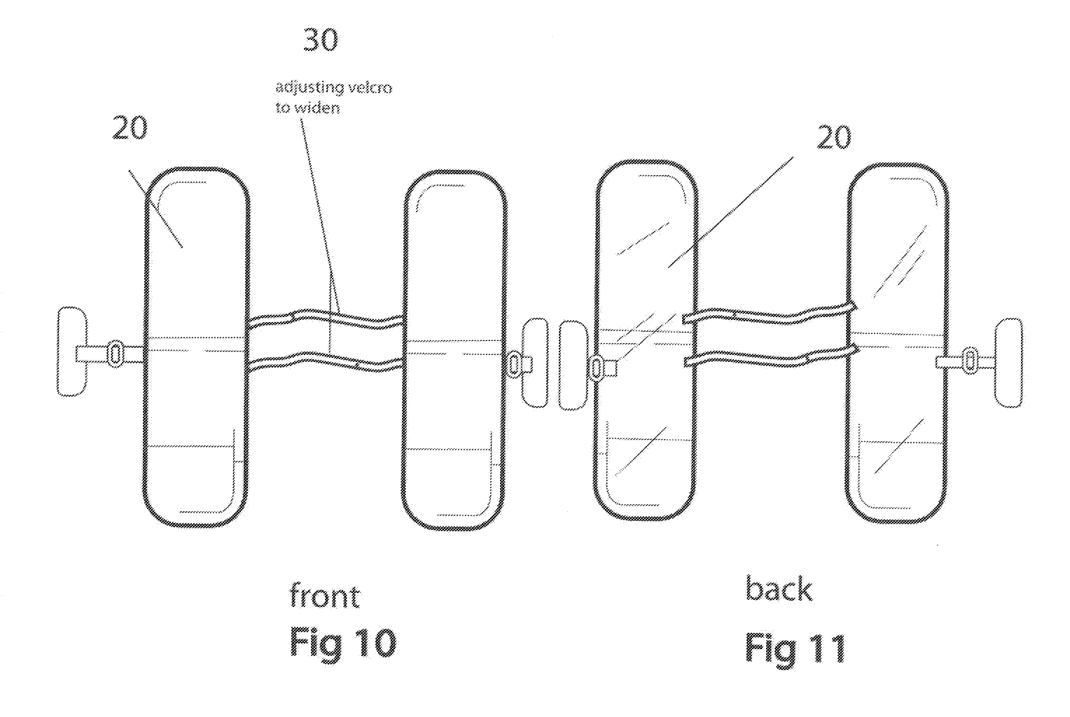

In alternative embodiment, the retainers (20, 21) consist of insert modules that allow the present invention to adjust to torso lengths of various types. In yet alternative embodiment as shown in FIG. 9, the pillow (5) can consist of inserts (42) allows the assembly to adjust to lower backs of various sizes. As shown in FIG. 10, 11 one or more attaching elements (30) can be used to attach the retainers together to support the length of the human torso while in the sleeping position. The attaching elements (30) between the opposing retainers can have adjustable attachments which allow the user to adjust the distance between the opposing retainers while sleeping.

What is claimed is:

1. An orthopedic pillow assembly to support an arc of a user's lower back:
the pillow assembly comprising:
a pillow having an inner core that is made of a firm flexible material;
the pillow having a length, width, and depth fabricated equivalent to support the arc of the user's body;
a first female fastener attached to an external portion of a first side of the pillow;
a second female fastener attached to an external portion directly opposite the first side the pillow;
a first and a second opposing retainer fabricated to a length equivalent to the torso of the users body;
the first and second opposing retainer having an inner body that is made of a firm flexible material;
a first and a second male fastener attached to an inner surface of the first and second opposing retainer respectively;
the female fastener attaches to the first male fastener;
the second female fastener attaches to the second male fastener to create a support area therebetween wherein the arc of the user's back is maintained in a straight state while the user lye within the support area.

2. The pillow assembly of claim 1 wherein the inner core has a circular, oval or polygonal shape.

3. The pillow assembly of claim 1 wherein the opposing retainers have a circular, oval or polygonal shape.

4. The pillow assembly of claim 1 wherein the inner core is made of a firm foam material.

5. The pillow assembly of claim 1 wherein the first and second retainer is made of a firm foam material.

6. The pillow assembly of claim 1 wherein the

[symbol]

7. The pillow assembly of claim 1 the inner core further comprises:
the inner core segmented into a first section and an opposite second section;
a pillow insert fabricated with a length equivalent to the inner core;
the pillow insert fabricated with an extension width to support the user's body;
the pillow insert having a first and a second attaching element attached to an external portion on opposing sides thereof;
the first section and the second section having a first and a second mating mechanism respectively attached to an internal peripheral section thereof; and the first mating mechanism and the second mating mechanism respectively coupled to the first and the second attaching element wherein the inner core of the pillow extends to support the are of the lower back of the user's body.

8. The pillow assembly of claim 1 the opposing retainers further comprises:
each opposing retainer segmented into a first section and an opposite second section;
a retainer insert fabricated with a width equivalent to each retainer;
the retainer insert fabricated with an extension length to support the user's body;
the retainer insert having a first and a second attaching element attached to an external portion on opposing sides thereof;
the first section and the second section of each retainer having a first and a second mating mechanism respectively attached to an internal peripheral section thereof; and the first mating mechanism and the second mating mechanism respectively coupled to the first and the second attaching element wherein each retainer extends to support the torso of the lower back of the user's body.
